(19) United States
(12) Patent Application Publication
(10) Pub. No.: US 2026/0212494 A1
(43) Pub. Date: Jul. 23, 2026

(54) SYSTEM AND METHOD FOR ANALYZING BODILY SAMPLES

(71) Applicant: S..D. Sight Diagnostics Ltd., Tel Aviv (IL)

(72) Inventors: Niv Steven Samuel MASTBOIM, Haifa (IL); Yochay Shlomo ESHEL, Sde Warburg (IL); Amir ZAIT, Binyamina (IL); Gil HERRMANN, Modi'in-Maccabim-Reut (IL); Eran RUBENS, Haifa (IL); Yishai AVIOR, Tel Aviv (IL)

(73) Assignee: S..D. Sight Diagnostics Ltd., Tel Aviv (IL)

(21) Appl. No.: 19/137,895

(22) PCT Filed: Dec. 11, 2023
(86) PCT No.: PCT/IB2023/062469

§ 371 Date: Jun. 11, 2025

(30) Foreign Application Priority Data
Dec. 12, 2022 (GB) 2218615.9

(51) Int. Cl. G06T 7/00; G01N 33/49; G02B 21/36

(52) U.S. Cl. CPC G06T 7/0012; G01N 33/49; G02B 21/367; G06T 2207/10056

(57) ABSTRACT

Apparatus and methods are described for use with a bodily sample. A plurality of microscopic images of the sample are acquired, using respective imaging modalities. The microscopic images are analyzed to identify and classify components within the bodily sample, and any anomalies within the sample that require further analysis are detected. In response to detecting that there are anomalies within the sample that require further analysis, a display image is generated that combines the images that were acquired using respective imaging modalities. In response to detecting that there are no anomalies within the sample that require further analysis, an output is generated based upon the analysis of the microscopic images, without generating a display image that combines images that were acquired using respective imaging modalities. Other applications are also described.

### CROSS-REFERENCES TO RELATED APPLICATIONS

The present application claims priority from GB Patent Application No. 2218615.9 to Mastboim, filed Dec. 12, 2022, entitled “System and method for analyzing bodily samples,” which is incorporated herein by reference.

### FIELD OF EMBODIMENTS OF THE INVENTION

Some applications of the presently disclosed subject matter relate generally to analysis of bodily samples, and in particular, to optical density and microscopic measurements that are performed upon blood samples.

### BACKGROUND

In some optics-based methods (e.g., diagnostic, and/or analytic methods), a property of a bodily sample, such as a blood sample, is determined by performing an optical measurement. For example, the density of a component (e.g., a count of the component per unit volume) may be determined by counting the component within a microscopic image. Similarly, the concentration and/or density of a component may be measured by performing optical absorption, transmittance, fluorescence, and/or luminescence measurements upon the sample. Typically, the sample is placed into a sample carrier and the measurements are performed with respect to a portion of the sample that is contained within a chamber of the sample carrier. The measurements that are performed upon the portion of the sample that is contained within the chamber of the sample carrier are analyzed in order to determine a property of the sample.

### SUMMARY OF EMBODIMENTS

In accordance with some applications of the present invention, a computer processor analyzes microscopic images and/or other data (e.g., optical absorption measurements, scattering measurements, and/or spectroscopic measurements) relating to a bodily sample, in order to determine properties of the sample. Typically the bodily sample is a blood sample. Alternatively or additionally, the bodily sample is a blood, urine, peritoneal fluid, cerebrospinal fluid, saliva, semen, sweat, sputum, vaginal fluid, stool, breast milk, bronchoalveolar lavage, gastric lavage, tears and/or nasal discharge sample. Typically, the computer processor analyzes a plurality of different microscopic images that are acquired using respective imaging modalities. For example, the microscope images may be acquired using fluorescent and brightfield imaging modalities. For some applications, the computer processor is configured to automatically identify components in blood such as platelets, white blood cells, anomalous white blood cells, circulating tumor cells, red blood cells, reticulocytes, Howell-Jolly bodies, etc. For some applications, the computer processor is configured to determine parameters relating to one or more of the components. For example, in relation to red blood cells, the computer processor determines parameters such as corpuscular hemoglobin (MCH), mean corpuscular volume (MCV), red blood cell distribution width (RDW), red blood cell morphologic features, clumping, and/or red blood cell abnormalities. Alternatively or additionally, in relation to white blood cells, the computer processor determines parameters such as absolute and relative numbers of neutrophils, lymphocytes, monocytes, eosinophils and basophils. For some applications, the computer processor performs normal and abnormal leukocyte differentiation, including detecting the existence of immature or hyper segmented cells, white blood cell agglutination or fragmentation, blasts, and/or atypical or abnormal lymphocytes. Further alternatively or additionally, in relation to platelets the computer processor determines parameters such as the presence of giant platelets, platelets clumps or abnormal platelets distribution, immature (i.e., reticulated) platelets fraction, average platelet size (MPV), platelet distribution width (PDW), platelet clumping, and/or platelet activation levels.

For some applications, the computer processor performs the above-described analysis of a sample. If the computer processor performs the analysis and does not detect any anomalies within the sample, then the computer processor generates an output based on the analysis, which in the case of a blood sample will typically be a complete blood count report. In such cases, the computer processor typically does not generate a display image of the sample. For some applications, in response to detecting an anomaly within the sample, then a display image of the sample is generated. As described in further detail hereinbelow, a display image is typically an image that combines microscopic images acquired using respective imaging modalities and that is displayed to a user for analysis, rather than a raw microscopic image of the type that is analyzed by the computer processor. Typically, the image that is generated has a similar appearance to that of a color smear image (e.g., similar to an image generated using Giemsa, Romanowsky-Giemsa, Wright-Romanowsky, Jenner, Wright, Field, May-Grünwald, methylene blue, new methylene blue, or Leishman smear staining), but without requiring a smear to be generated from the sample. Typically, the computer processor sends data to a remote computer server and the remote computer processor generates the display image using the data. Alternatively, the display image is generated by a local computer processor and is sent to the remote computer network.

For some applications, the display image is generated in response to detecting a distributional anomaly (i.e., an anomaly relating to the number of a type of entity distributed within the sample). Alternatively or additionally, the display image is generated is performed in response to detecting a morphological anomaly (i.e., an anomaly relating to one or more occurrences of a given morphological entity or phenomenon within the sample). For some applications, in response to detecting an anomaly, the sample is first flagged, such that the user can select whether they wish to see an image of the sample.

For some applications, the display image is generated in response to detecting one or more of the following entities (and/or more than a given concentration thereof): platelet clumps, giant platelets, enlarged platelets, blast cell, immature granulocytes, atypical lymphocytes, abnormal lymphocytes, nucleated red blood cells, schistocytes, sickle cells, target cells, tear drop cells, Howell Jolly bodies, Pappenheimer bodies, basophilic stippling, and/or parasites. For some applications, the display image is generated in response to detecting a color abnormality, (e.g., a red blood cell color abnormality, e.g., polychromatic red blood cells and/or hypochromatic red blood cells), a size abnormality (e.g., red blood cell macrocytosis, microcytosis, and/or anisocytosis), and/or a shape abnormality (e.g., poikilocytosis, target cells, schistocytes, helmet cells, sickle cells, spherocytes, elliptocytes, ovalocytes, tear drop cells, stomatocytes, acanthocytes, and/or echinocytes).

It is noted that, typically, the display image is generated using the same data that are used to perform the analysis of the sample. Typically, the display image is formed by combining a plurality of microscopic images that were acquired using respective imaging modalities, such as the brightfield and/or fluorescent imaging modalities described hereinabove. Typically, the image that is generated has a similar appearance to that of a color smear image (e.g., similar to an image generated using Giemsa, Romanowsky-Giemsa, Wright-Romanowsky, Jenner, Wright, Field, May-Grünwald, methylene blue, new methylene blue, or Leishman smear staining), but without requiring the sample to be smeared and/or stained. This is in contrast to alternative techniques, whereby in response to an optical measurement unit detecting an anomaly within the sample, the subject is instructed to take a further sample of blood and image the further sample with a different optical measurement system (that typically requires the sample to be smeared and/or stained) in order to generate a display image of the blood. Rather, in accordance with the present disclosure, a display image is generated using the same data and the same sample as were analyzed in the previous step. However, those data are used to generate a display image rather than being analyzed to generate a report. Typically, this is less cumbersome for the subject and saves resources relative to the alternative techniques. For example, it is not necessary for a clinic to include two separate analysis systems, i.e., a first analysis system for performing the initial analysis and a second analysis system for imaging the sample, in response to the first system detecting an anomaly within the sample. In addition, the subject is only required to take one sample, and that same sample is used both for performing the initial analysis and for generating the display image. It is noted however, that typically the display image is only generated in response to the computer processor detecting an anomaly within the sample. This is because, although all of the data that are required for generating the display image have typically already been acquired in the earlier step, it requires additional computational resources to generate the display image. Therefore, the step of generating the display image is only performed if a need is identified, such as in the event of the computer processor detecting an anomaly within the sample.

There is therefore provided, in accordance with some applications of the present invention, a method for use with a bodily sample, the method including:
using a microscope, acquiring a plurality microscopic images of the sample, using respective imaging modalities; and
using at least one computer processor:
   analyzing the microscopic images to identify and classify components within the bodily sample;
   detecting whether there are any anomalies within the sample that require further analysis;
   in response to detecting that there are anomalies within the sample that require further analysis:
      generating a display image that combines the images that were acquired using respective imaging modalities; and
      driving a display to display the display image to a user; and
   in response to detecting that there are not anomalies within the sample that require further analysis, generating an output based upon the analysis of the microscopic images, without generating a display image that combines images that were acquired using respective imaging modalities.

In some applications, the bodily sample includes a blood sample and generating the output based upon the analysis of the microscopic images, without generating the display image that combines images that were acquired using respective imaging modalities, includes generating a complete blood count.

In some applications, driving a display to display the display image to a user includes driving a display to display the display image to a user at a location that is remote from the microscope.

In some applications,

the method further includes, in response to detecting that there are anomalies within the sample that require further analysis, outputting an indication of the anomalies to the user, and

generating the display image that combines the images that were acquired using respective imaging modalities includes generating the display image that combines the images that were acquired using respective imaging modalities in response to receiving an input from the user indicating that the display image should be generated.

In some applications, generating the display image that combines the images that were acquired using respective imaging modalities includes generating an image having a similar appearance to that of a color smear image without requiring a smear to be generated from the sample.

In some applications, acquiring a plurality microscopic images of the sample, using respective imaging modalities includes acquiring at least some fluorescent microscopic images, and generating the display image that combines the images that were acquired using respective imaging modalities includes generating a display image that incorporates the fluorescent microscopic images.

In some applications, detecting the anomaly includes detecting an indication that a concentration of a given entity within the sample is greater than a threshold amount.

In some applications, generating the display image includes generating a display image showing the given entity and/or generating a display image in which the given entity is highlighted.

In some applications, the bodily sample includes a blood sample and detecting the anomaly includes detecting an anomaly relating to platelets.

In some applications, detecting the anomaly includes detecting an indication of a presence, or a concentration that exceeds a threshold, of giant platelets, platelet clumps, and/or enlarged platelets.

In some applications, the bodily sample includes a blood sample and detecting the anomaly includes detecting an anomaly relating to white blood cells.

In some applications, detecting the anomaly includes detecting an indication of a presence, or a concentration that exceeds a threshold, of blast cells, immature granulocytes, atypical lymphocytes, and/or abnormal lymphocytes.

In some applications, the bodily sample includes a blood sample and detecting the anomaly includes detecting an anomaly relating to red blood cells.

In some applications, detecting the anomaly includes detecting an indication of a presence, or a concentration that exceeds a threshold, of nucleated red blood cells, schistocytes, sickle cells, target cells, and/or tear drop cells.

In some applications, the bodily sample includes a blood sample and detecting the anomaly includes detecting a color abnormality.

In some applications, detecting the anomaly includes detecting an indication of a presence, or a concentration that exceeds a threshold, of polychromatic red blood cells and/or hypochromatic red blood cells.

In some applications, the bodily sample includes a blood sample and detecting the anomaly includes detecting a size abnormality.

In some applications, detecting the anomaly includes detecting an indication of a presence, or a concentration that exceeds a threshold, of red blood cell macrocytosis, microcytosis, and/or anisocytosis.

In some applications, the bodily sample includes a blood sample and detecting the anomaly includes detecting a shape abnormality.

In some applications, detecting the anomaly includes detecting an indication of a presence, or a concentration that exceeds a threshold, of poikilocytosis, target cells, schistocytes, helmet cells, sickle cells, spherocytes, elliptocytes, ovalocytes, tear drop cells, stomatocytes, acanthocytes, and/or echinocytes.

There is further provided, in accordance with some applications of the present invention, apparatus for use with a bodily sample, the apparatus including:
a microscope configured to acquire a plurality microscopic images of the sample, using respective imaging modalities; and
at least one computer processor configured to:
   analyze the microscopic images to identify and classify components within the bodily sample;
   detect whether there are any anomalies within the sample that require further analysis;
   in response to detecting that there are anomalies within the sample that require further analysis:
      generate a display image that combines the images that were acquired using respective imaging modalities; and
      drive a display to display the display image to a user; and
   in response to detecting that there are not anomalies within the sample that require further analysis, generate an output based upon the analysis of the microscopic images, without generating a display image that combines images that were acquired using respective imaging modalities.

In some applications, the bodily sample includes a blood sample and the at least one computer processor is configured to generate the output based upon the analysis of the microscopic images, without generating the display image that combines images that were acquired using respective imaging modalities, by generating a complete blood count.

In some applications, the at least one computer processor is configured to drive a display to display the display image to a user at a location that is remote from the microscope.

In some applications, the at least one computer processor is configured to generate the display image that combines the images that were acquired using respective imaging modalities by generating an image having a similar appearance to that of a color smear image without requiring a smear to be generated from the sample.

In some applications, the microscope is configured to acquire at least some fluorescent microscopic images, and the at least one computer processor is configured to generate the display image that combines the images that were acquired using respective imaging modalities by generating a display image that incorporates the fluorescent microscopic images.

There is further provided, in accordance with some applications of the present invention, a computer software product, for use with a plurality microscopic images of a bodily sample, each of the microscopic images having been acquired using respective imaging modalities, the computer software product including a non-transitory computer-readable medium in which program instructions are stored, which instructions, when read by a computer cause the computer to perform the steps of:
analyzing the microscopic images to identify and classify components within the bodily sample;
detecting whether there are any anomalies within the sample that require further analysis;
in response to detecting that there are anomalies within the sample that require further analysis:
generating a display image that combines the images that were acquired using respective imaging modalities; and
driving a display to display the display image to a user; and
in response to detecting that there are not anomalies within the sample that require further analysis, generating an output based upon the analysis of the microscopic images, without generating a display image that combines images that were acquired using respective imaging modalities.

The present invention will be more fully understood from the following detailed description of embodiments thereof, taken together with the drawings, in which:

### BRIEF DESCRIPTION OF THE DRAWINGS

FIG. 1 is a block diagram showing components of a biological sample analysis system, in accordance some applications of the present invention;

FIGS. 2A, 2B, and 2C are schematic illustrations of an optical measurement unit, in accordance with some applications of the present invention;

FIGS. 3A, 3B, and 3C are schematic illustrations of respective views of a sample carrier that is used for performing both microscopic measurements and optical density measurements, in accordance with some applications of the present invention;

FIGS. 4A, 4B, 4C, and 4D are flowcharts showing steps of methods that are performed, in accordance with some applications of the present invention; and

FIGS. 5A and 5B are flowcharts showing steps of workflows that are performed using a computer network, in accordance with some applications of the present invention.

### DETAILED DESCRIPTION OF EMBODIMENTS

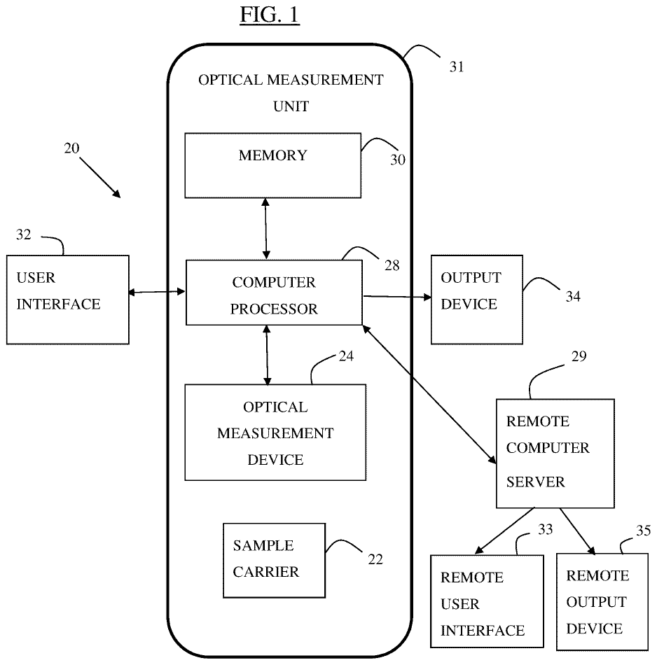

Reference is now made to FIG. 1, which is block diagram showing components of a bodily sample analysis system 20, in accordance with some applications of the present invention. Typically, a bodily sample is placed into a sample carrier 22. Typically the bodily sample is a blood sample. Alternatively or additionally, the bodily sample is a blood, urine, peritoneal fluid, cerebrospinal fluid, saliva, semen, sweat, sputum, vaginal fluid, stool, breast milk, bronchoalveolar lavage, gastric lavage, tears and/or nasal discharge sample. While the sample is disposed in the sample carrier, optical measurements are performed upon the sample using one or more optical measurement devices 24. For example, the optical measurement devices may include a microscope (e.g., a digital microscope), a spectrophotometer, a photometer, a spectrometer, a camera, a spectral camera, a hyperspectral camera, a fluorometer, a spectrofluorometer, and/or a photodetector (such as a photodiode, a photoresistor, and/or a phototransistor). For some applications, the optical measurement devices include dedicated light sources (such as light emitting diodes, incandescent light sources, etc.) and/or optical elements for manipulating light collection and/or light emission (such as lenses, diffusers, filters, etc.).

A computer processor 28 typically receives and processes optical measurements that are performed by the optical measurement device. Further typically, the computer processor controls the acquisition of optical measurements that are performed by the one or more optical measurement devices. The computer processor communicates with a memory 30. A user (e.g., a laboratory technician, or an individual from whom the sample was drawn) sends instructions to the computer processor via a user interface 32. For some applications, the user interface includes a keyboard, a mouse, a joystick, a touchscreen device (such as a smartphone or a tablet computer), a touchpad, a trackball, a voice-command interface, and/or other types of user interfaces that are known in the art. Typically, the computer processor generates an output via an output device 34. Further typically, the output device includes a display, such as a monitor, and the output includes an output that is displayed on the display. For some applications, the processor generates an output on a different type of visual, text, graphics, tactile, audio, and/or video output device, e.g., speakers, headphones, a smartphone, or a tablet computer. For some applications, user interface 32 acts as both an input interface and an output interface, i.e., it acts as an input/output interface. For some applications, the processor generates an output on a computer-readable medium (e.g., a non-transitory computer-readable medium), such as a disk, or a portable USB drive, and/or generates an output on a printer.

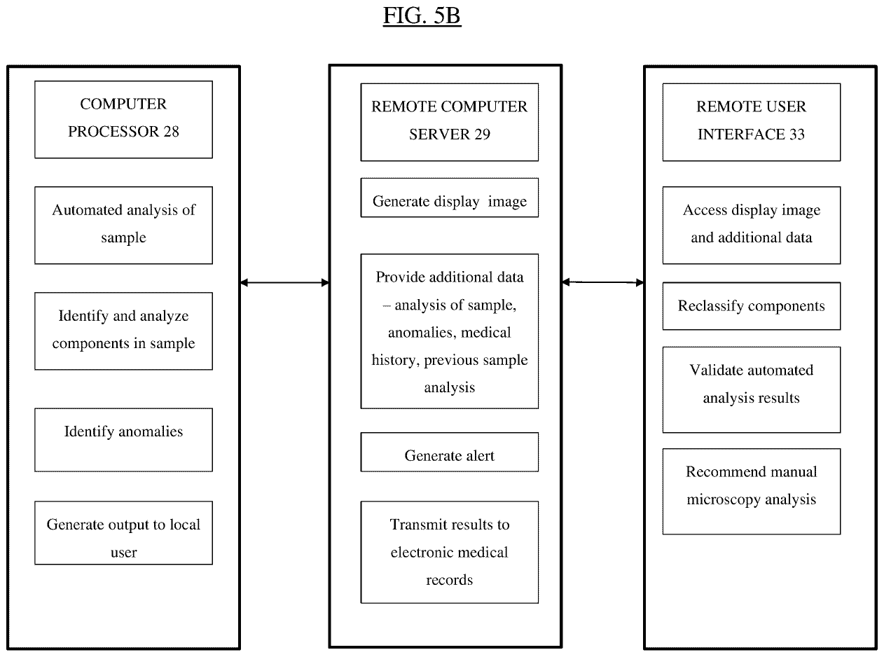

For some applications, computer processor 28 communicates with a remote computer processor or computer network, e.g., a remote (cloud-based) computer server 29, for example, via a communications network. For some applications, the remote computer server generates outputs and/or receives inputs from a remote user interface 33. For some applications, the remote user interface includes a keyboard, a mouse, a joystick, a touchscreen device (such as a smartphone or a tablet computer), a touchpad, a trackball, a voice-command interface, and/or other types of user interfaces that are known in the art. For some applications, the remote computer server generates outputs on a remote output device 35. Further typically, the output device includes a display, such as a monitor, and the output includes an output that is displayed on the display. For some applications, the processor generates an output on a different type of visual, text, graphics, tactile, audio, and/or video output device, e.g., speakers, headphones, a smartphone, or a tablet computer. For some applications, remote user interface 33 acts as both an input interface and an output interface, i.e., it acts as an input/output interface. For some applications, the processor generates an output on a computer-readable medium (e.g., a non-transitory computer-readable medium), such as a disk, or a portable USB drive, and/or generates an output on a printer. For some applications, the remote user interface and/or the remote output unit is itself disposed remotely from the remote computer server, and a remote user communicates with the remote computer server via a communications network. For example, the remote user interface may be a computer, a smartphone, or a tablet computer, through which a remote user communicates with the remote computer server via a communications network. Some aspects of the communication between computer processor and the remote computer server are described in further detail hereinbelow with reference to FIGS. 5A and 5B.

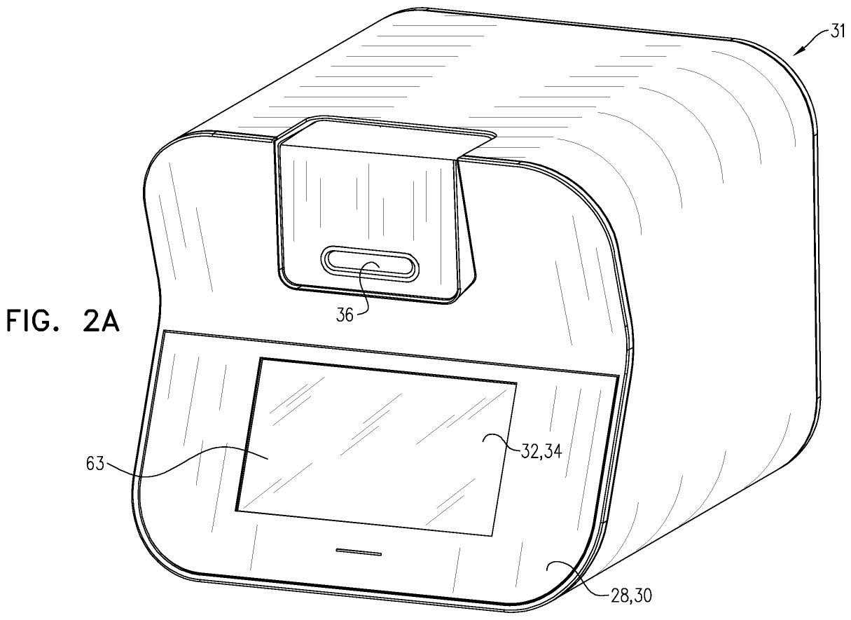

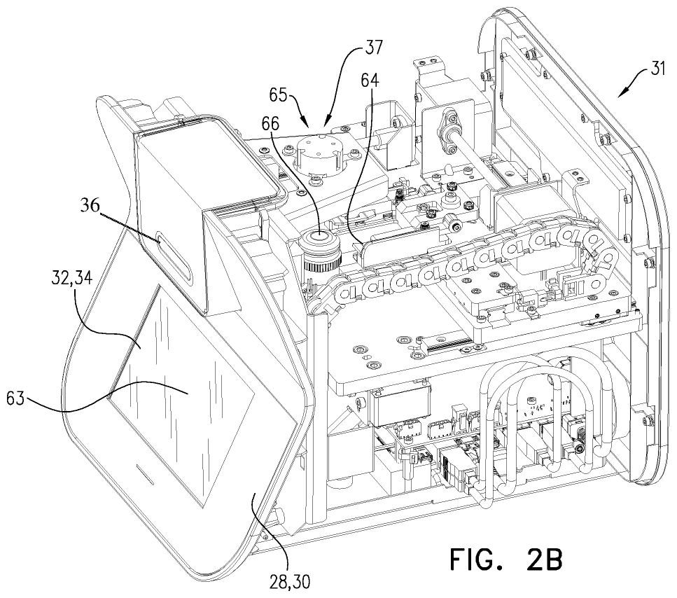

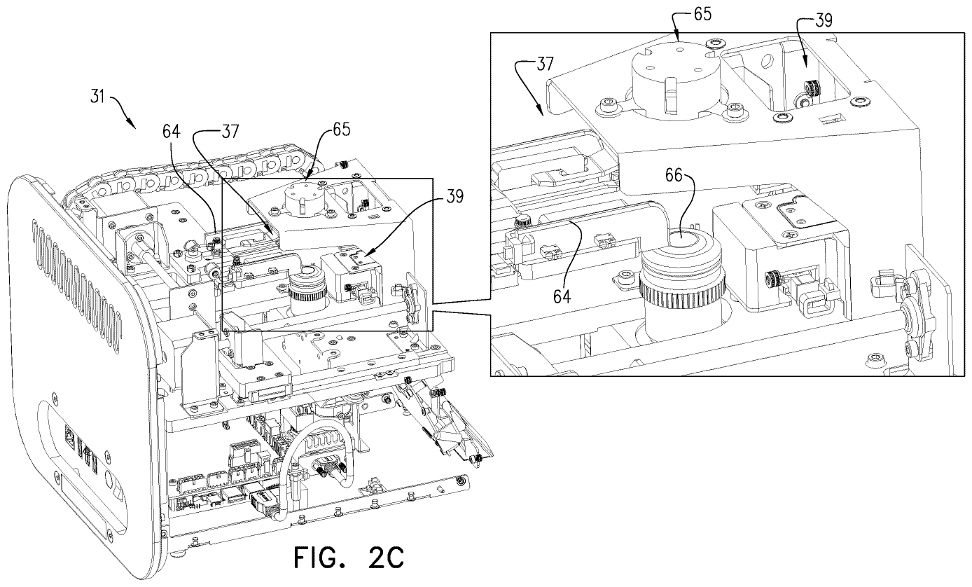

Reference is now made to FIGS. 2A, 2B, and 2C, which are schematic illustrations of an optical measurement unit 31, in accordance with some applications of the present invention. FIG. 2A shows an oblique view of the exterior of the fully assembled device, while FIGS. 2B and 2C shows respective oblique views of the device with the cover having been made transparent, such that components within the device are visible. For some applications, one or more optical measurement devices 24 (and/or computer processor 28 and memory 30) is housed inside optical measurement unit 31. In order to perform the optical measurements upon the sample, sample carrier 22 is placed inside the optical measurement unit. For example, the optical measurement unit may define a slot 36, via which the sample carrier is inserted into the optical measurement unit. Typically, the optical measurement unit includes a stage 64, which is configured to support sample carrier 22 within the optical measurement unit. For some applications, a screen 63 on the cover of the optical measurement unit (e.g., a screen on the front cover of the optical measurement unit, as shown) functions as user interface 32 and/or output device 34.

Typically, the optical measurement unit includes microscope system 37 (shown in FIGS. 2B-C) configured to perform microscopic imaging of a portion of the sample. For some applications, the microscope system includes a set of light sources 65 (which typically include a set of brightfield light sources (e.g. light emitting diodes) that are configured to be used for brightfield imaging of the sample, a set of fluorescent light sources (e.g. light emitting diodes) that are configured to be used for fluorescent imaging of the sample), and a camera (e.g., a CCD camera, or a CMOS camera) configured to image the sample. Typically, the optical measurement unit also includes an optical-density-measurement unit 39 (shown in FIG. 2C) configured to perform optical measurements (e.g., optical absorption measurements, scattering measurements, and/or spectroscopic measurements) on a second portion of the sample. For some applications, the optical-density-measurement unit includes a set of optical-density-measurement light sources (e.g., light emitting diodes) and light detectors, which are configured for performing optical density measurements on the sample. For some applications, each of the aforementioned sets of light sources (i.e., the set of brightfield light sources, the set of fluorescent light sources, and the set optical-density-measurement light sources) includes a plurality of light sources (e.g. a plurality of light emitting diodes), each of which is configured to emit light at a respective wavelength or at a respective band of wavelengths.

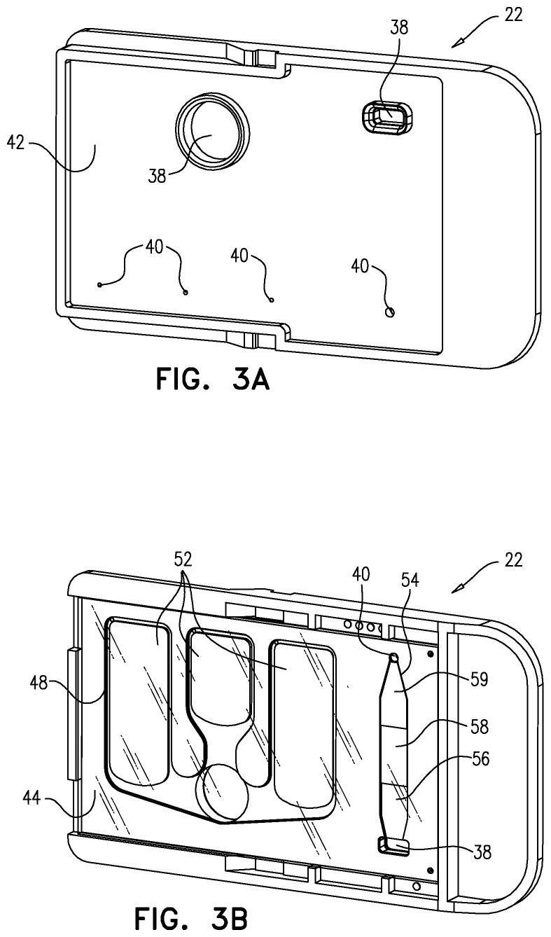

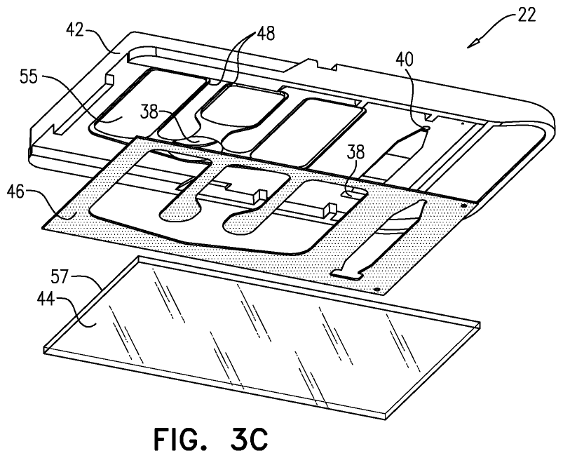

Reference is now made to FIGS. 3A and 3B, which are schematic illustrations of respective views of sample carrier 22, in accordance with some applications of the present invention. FIG. 3A shows a top view of the sample carrier (the top cover of the sample carrier being shown as being opaque in FIG. 3A, for illustrative purposes), and FIG. 3B shows a bottom view (in which the sample carrier has been rotated around its short edge with respect to the view shown in FIG. 3A). Typically, the sample carrier includes a first set 52 of one or more sample chambers, which are used for performing microscopic analysis upon the sample, and a second set 54 of sample chambers, which are used for performing optical density measurements upon the sample. Typically, the sample chambers of the sample carrier are filled with a bodily sample, such as blood via sample inlet holes 38. For some applications, the sample chambers define one or more outlet holes 40. The outlet holes are configured to facilitate filling of the sample chambers with the bodily sample, by allowing air that is present in the sample chambers to be released from the sample chambers. Typically, as shown, the outlet holes are located longitudinally opposite the inlet holes (with respect to a sample chamber of the sample carrier). For some applications, the outlet holes thus provide a more efficient mechanism of air escape than if the outlet holes were to be disposed closer to the inlet holes.

Reference is made to FIG. 3C, which shows an exploded view of sample carrier 22, in accordance with some applications of the present invention. For some applications, the sample carrier includes at least three components: a molded component 42, a glass layer 44 (e.g., a glass sheet), and an adhesive layer 46 configured to adhere the glass layer to an underside of the molded component. The molded component is typically made of a polymer (e.g., a plastic) that is molded (e.g., via injection molding) to provide the sample chambers with a desired geometrical shape. For example, as shown, the molded component is typically molded to define inlet holes 38, outlet holes 40, and gutters 48 which surround the central portion of each of the sample chambers. The gutters typically facilitate filling of the sample chambers with the bodily sample, by allowing air to flow to the outlet holes, and/or by allowing the bodily sample to flow around the central portion of the sample chamber.

For some applications, a sample carrier as shown in FIGS. 3A-C is used when performing a complete blood count on a blood sample. For some such applications, the sample carrier is used with optical measurement unit 31 configured as generally shown and described with reference to FIGS. 2A-C. For some applications, a first portion of the blood sample is placed inside first set 52 of sample chambers (which are used for performing microscopic analysis upon the sample, e.g., using microscope system 37 (shown in FIGS. 2B-C)), and a second portion of the blood sample is placed inside second set 54 of sample chambers (which are used for performing optical density measurements upon the sample, e.g., using optical-density-measurement unit 39 (shown in FIG. 2C)). For some applications, first set 52 of sample chambers includes a plurality of sample chambers, while second set 54 of sample chambers includes only a single sample chamber, as shown. However, the scope of the present application, includes using any number of sample chambers (e.g., a single sample chamber or a plurality of sample chambers) within either the first set of sample chambers or within the second set of sample chambers, or any combination thereof. The first portion of the blood sample is typically diluted with respect to the second portion of the blood sample. For example, the diluent may contain pH buffers, stains, fluorescent stains, antibodies, sphering agents, lysing agents, etc. Typically, the second portion of the blood sample, which is placed inside second set 54 of sample chambers is a natural, undiluted blood sample. Alternatively or additionally, the second portion of the blood sample may be a sample that underwent some modification, including, for example, one or more of dilution (e.g., dilution in a controlled fashion), addition of a component or reagent, or fractionation.

For some applications, one or more staining substances are used to stain the first portion of the blood sample (which is placed inside first set 52 of sample chambers) before the sample is imaged microscopically. For example, the staining substance may be configured to stain DNA with preference over staining of other cellular components. Alternatively, the staining substance may be configured to stain all cellular nucleic acids with preference over staining of other cellular components. For example, the sample may be stained with Acridine Orange reagent, Hoechst reagent, and/or any other staining substance that is configured to preferentially stain DNA and/or RNA within the blood sample. Optionally, the staining substance is configured to stain all cellular nucleic acids but the staining of DNA and RNA are each more prominently visible under some lighting and filter conditions, as is known, for example, for Acridine Orange. Images of the sample may be acquired using imaging conditions that allow detection of cells (e.g., brightfield) and/or imaging conditions that allow visualization of stained bodies (e.g. appropriate fluorescent illumination). Typically, the first portion of the sample is stained with Acridine Orange and with a Hoechst reagent. For example, the first (diluted) portion of the blood sample may be prepared using techniques as described in U.S. Pat. No. 9,329,129 to Pollak, which is incorporated herein by reference, and which describes a method for preparation of blood samples for analysis that involves a dilution step, the dilution step facilitating the identification and/or counting of components within microscopic images of the sample. For some applications, the first portion of the sample is stained with one or more stains that cause platelets within the sample to be visible under brightfield imaging conditions and/or under fluorescent imaging conditions, e.g., as described hereinabove. For example, the first portion of the sample may be stained with methylene blue, new methylene blue, and/or Romanowsky stains.

Referring again to FIG. 2B, typically, sample carrier 22 is supported within the optical measurement unit by stage 64. Further typically, the stage has a forked design, such that the sample carrier is supported by the stage around the edges of the sample carrier, but such that the stage does not interfere with the visibility of the sample chambers of the sample carrier by the optical measurement devices. For some applications, the sample carrier is held within the stage, such that molded component 42 of the sample carrier is disposed above the glass layer 44, and such that an objective lens 66 of a microscope unit of the optical measurement unit is disposed below the glass layer of the sample carrier. Typically, at least some light sources 65 that are used during microscopic measurements that are performed upon the sample (for example, light sources that are used during brightfield imaging) illuminate the sample carrier from above the molded component. Further typically, at least some additional light sources (not shown) illuminate the sample carrier from below the sample carrier (e.g., via the objective lens). For example, light sources that are used to excite the sample during fluorescent microscopy may illuminate the sample carrier from below the sample carrier (e.g., via the objective lens).

Typically, prior to being imaged microscopically, the first portion of blood (which is placed in first set 52 of sample chambers) is allowed to settle such as to form a monolayer of cells, e.g., using techniques as described in U.S. Pat. No. 9,329,129 to Pollak, which is incorporated herein by reference. For some applications, the first portion of blood is a cell suspension and the chambers belonging to the first set 52 of chambers each define a cavity 55 that includes a base surface 57 (shown in FIG. 3C). Typically, the cells in the cell suspension are allowed to settle on the base surface of the sample chamber of the carrier to form a monolayer of cells on the base surface of the sample chamber. Subsequent to the cells having been left to settle on the base surface of the sample chamber (e.g., by having been left to settle for a predefined time interval), at least one microscopic image of at least a portion of the monolayer of cells is typically acquired. Typically, a plurality of images of the monolayer are acquired, each of the images corresponding to an imaging field that is located at a respective, different area within the imaging plane of the monolayer. Typically, an optimum depth level at which to focus the microscope in order to image the monolayer is determined, e.g., using techniques as described in U.S. Pat. No. 10,176,565 to Greenfield, which is incorporated herein by reference. For some applications, respective imaging fields have different optimum depth levels from each other.

It is noted that, in the context of the present application, the term monolayer is used to mean a layer of cells that have settled, such as to be disposed within a single focus level of the microscope. Within the monolayer there may be some overlap of cells, such that within certain areas there are two or more overlapping layers of cells. For example, red blood cells may overlap with each other within the monolayer, and/or platelets may overlap with, or be disposed above, red blood cells within the monolayer.

For some applications, the microscopic analysis of the first portion of the blood sample is performed with respect to the monolayer of cells. Typically, the first portion of the blood sample is imaged under brightfield imaging, i.e., under illumination from one or more light sources (e.g., one or more light emitting diodes, which typically emit light at respective spectral bands). Further typically, the first portion of the blood sample is additionally imaged under fluorescent imaging. Typically, the fluorescent imaging is performed by exciting stained objects (i.e., objects that have absorbed the stain(s)) within the sample by directing light toward the sample at known excitation wavelengths (i.e., wavelengths at which it is known that stained objects emit fluorescent light if excited with light at those wavelengths), and detecting the fluorescent light. Typically, for the fluorescent imaging, a separate set of light sources (e.g., one or more light emitting diodes) is used to illuminate the sample at the known excitation wavelengths.

As described with reference to US 2019/0302099 to Pollak, which is incorporated herein by reference, for some applications, sample chambers belonging to set 52 (which is used for microscopy measurements) have different heights from each other, in order to facilitate different measurands being measured using microscope images of respective sample chambers, and/or different sample chambers being used for microscopic analysis of respective sample types. For example, if a blood sample, and/or a monolayer formed by the sample, has a relatively low density of red blood cells, then measurements may be performed within a sample chamber of the sample carrier having a greater relative height (i.e., a sample chamber of the sample carrier having a greater height relative to a different sample chamber having a lower relative height), such that there is a sufficient density of cells, and/or such that there is a sufficient density of cells within the monolayer formed by the sample, to provide statistically reliable data. Such measurements may include, for example red blood cell density measurements, measurements of other cellular attributes, (such as counts of abnormal red blood cells, red blood cells that include intracellular bodies (e.g., pathogens, Howell-Jolly bodies), etc.), and/or hemoglobin concentration. Conversely, if a blood sample, and/or a monolayer formed by the sample, has a relatively high density of red blood cells, then such measurements may be performed upon a sample chamber of the sample carrier having a lower relative height, for example, such that there is a sufficient sparsity of cells, and/or such that there is a sufficient sparsity of cells within the monolayer of cells formed by the sample, that the cells can be identified within microscopic images. For some applications, such methods are performed even without the variation in height between the sample chambers belonging to set 52 being precisely known.

For some applications, based upon the measurand that is being measured, the sample chamber within the sample carrier upon which to perform optical measurements is selected. For example, a sample chamber of the sample carrier having a greater height may be used to perform a white blood cell count (e.g., to reduce statistical errors which may result from a low count in a shallower region), white blood cell differentiation, and/or to detect more rare forms of white blood cells. Conversely, in order to determine mean corpuscular hemoglobin (MCH), mean corpuscular volume (MCV), red blood cell distribution width (RDW), red blood cell morphologic features, and/or red blood cell abnormalities, microscopic images may be obtained from a sample chamber of the sample carrier having a lower relative height, since in such sample chambers the cells are relatively sparsely distributed across the area of the region, and/or form a monolayer in which the cells are relatively sparsely distributed. Similarly, in order to count platelets, classify platelets, and/or extract any other attributes (such as volume) of platelets, microscopic images may be obtained from a sample chamber of the sample carrier having a lower relative height, since within such sample chambers there are fewer red blood cells which overlap (fully or partially) with the platelets in microscopic images, and/or in a monolayer.

In accordance with the above-described examples, it is preferable to use a sample chamber of the sample carrier having a lower relative height for performing optical measurements for measuring some measurands within a sample (such as a blood sample), whereas it is preferable to use a sample chamber of the sample carrier having a greater relative height for performing optical measurements for measuring other measurands within such a sample. Therefore, for some applications, a first measurand within a sample is measured, by performing a first optical measurement upon (e.g., by acquiring microscopic images of) a portion of the sample that is disposed within a first sample chamber belonging to set 52 of the sample carrier, and a second measurand of the same sample is measured, by performing a second optical measurement upon (e.g., by acquiring microscopic images of) a portion of the sample that is disposed within a second sample chamber of set 52 of the sample carrier. For some applications, the first and second measurands are normalized with respect to each other, for example, using techniques as described in US 2019/0145963 to Zait, which is incorporated herein by reference.

Typically, in order to perform optical density measurements upon the sample, it is desirable to know the optical path length, the volume, and/or the thickness of the portion of the sample upon which the optical measurements were performed, as precisely as possible. Typically, an optical density measurement is performed on the second portion of the sample (which is typically placed into second set 54 of sample chambers in an undiluted form). For example, the concentration and/or density of a component may be measured by performing optical absorption, transmittance, fluorescence, scattering, spectroscopic, and/or luminescence measurements upon the sample.

Referring again to FIG. 3B, for some applications, sample chambers belonging to set 54 (which is used for optical density measurements), typically define at least a first region 56 (which is typically deeper) and a second region 58 (which is typically shallower), the height of the sample chambers varying between the first and second regions in a predefined manner, e.g., as described in US 2019/0302099 to Pollak, which is incorporated herein by reference. The heights of first region 56 and second region 58 of the sample chamber are defined by a lower surface that is defined by the glass layer and by an upper surface that is defined by the molded component. The upper surface at the second region is stepped with respect to the upper surface at the first region. The step between the upper surface at the first and second regions, provides a predefined height difference Δh between the regions, such that even if the absolute height of the regions is not known to a sufficient degree of accuracy (for example, due to tolerances in the manufacturing process), the height difference Δh is known to a sufficient degree of accuracy to determine a parameter of the sample, using the techniques described herein, and as described in US 2019/0302099 to Pollak, which is incorporated herein by reference. For some applications, the height of the sample chamber varies from the first region 56 to the second region 58, and the height then varies again from the second region to a third region 59, such that, along the sample chamber, first region 56 defines a maximum height region, second region 58 defines a medium height region, and third region 59 defines a minimum height region. For some applications, additional variations in height occur along the length of the sample chamber, and/or the height varies gradually along the length of the sample chamber.

As described hereinabove, while the sample is disposed in the sample carrier, optical measurements are performed upon the sample using one or more optical measurement devices 24. Typically, the sample is viewed by the optical measurement devices via the glass layer, glass being transparent at least to wavelengths that are typically used by the optical measurement device. Typically, the sample carrier is inserted into optical measurement unit 31, which houses the optical measurement device while the optical measurements are performed. Typically, the optical measurement unit houses the sample carrier such that the molded layer is disposed above the glass layer, and such that the optical measurement unit is disposed below the glass layer of the sample carrier and is able to perform optical measurements upon the sample via the glass layer. The sample carrier is formed by adhering the glass layer to the molded component. For example, the glass layer and the molded component may be bonded to each other during manufacture or assembly (e.g. using thermal bonding, solvent-assisted bonding, ultrasonic welding, laser welding, heat staking, adhesive, mechanical clamping and/or additional substrates). For some applications, the glass layer and the molded component are bonded to each other during manufacture or assembly using adhesive layer 46.

For some microscopy applications, microscopic images of imaging fields are acquired using a plurality of different imaging modalities. For example, as described hereinabove, brightfield images may be acquired under illumination of the sample at several, respective, different wavelength bands. The brightfield images may be acquired while cells (e.g., a monolayer of the cells) are in focus or out of focus. Alternatively or additionally, fluorescent images are acquired by exciting stained objects (i.e., objects that have absorbed the stain(s)) within the sample by directing light toward the sample at known excitation wavelengths (i.e., wavelengths at which it is known that stained objects emit fluorescent light if excited with light at those wavelengths), and detecting the fluorescent light. Respective fluorescent images are acquired by exciting the sample with light at respective, different wavelength bands, or by exciting the sample with light a given wavelength band and then using emission filters that filter light that is emitted from the sample at respective wavelength bands.

Typically, the computer processor analyzes the microscopic images and/or other data relating to the sample (e.g., optical absorption measurements, scattering measurements, and/or spectroscopic measurements), in order to determine properties of the sample. For some applications, the computer processor additionally outputs images of the sample to a user via output device 34. It may be challenging though for a human observer to extract useful information from the images, especially if that information is contained in the overlap between images that were acquired using respective, different imaging modalities and these images are overlaid upon each other as black-and-white or grayscale images. For example, in order to verify that an element is an intraerythrocytic parasite, it may be helpful to see a single image in which the parasite candidate is visible and red blood cells are visible. The red blood cells are typically visible in brightfield images (e.g., brightfield images acquired under violet illumination), whereas the parasites are typically visible in fluorescent images.

Therefore, it is helpful to see such images overlaid upon each other, but in which elements from the respective imaging modalities are visible without interfering with each other. Similarly, in order to see morphological features of white blood cells (which can help in the classification of an element as a white blood cell, and/or as a given type of white blood cell), it is typically helpful to see respective fluorescent images acquired under respective fluorescent illumination conditions overlaid upon each other.

Therefore, in accordance with some applications of the present invention, a plurality of images of a microscopic imaging field of a blood sample are acquired, each of the images being acquired using respective, different imaging conditions. Typically, at least one of the images is a brightfield image that is acquired under violet lighting conditions (e.g., under lighting by light at a wavelength within the range of 400 nm-450 nm). For some applications, the brightfield image is an off-focus image that is acquired under violet lighting conditions. Further typically, at least one of the images is a fluorescent image. A computer processor combines data from each of the plurality of images such as to generate an artificial color microscopic image of the microscopic imaging field that appears like a color smear image. Typically, one or more color models such as RGB, CIE, HSV, and/or a combination thereof is used to generate the artificial color microscopic image.

Typically, the image that was acquired under brightfield, violet lighting conditions is mapped to a red channel of the artificial color microscopic image. Further typically, the image is converted to a negative contrast image before being mapped to the red channel. For some applications, the result of mapping to the negative contrast image of the image acquired under brightfield, violet lighting conditions is that red blood cells have a similar appearance to the appearance of red blood cells in a color smear image (e.g., similar to those generated using Giemsa, Romanowsky-Giemsa, Wright-Romanowsky, Jenner, Wright, Field, May-Grünwald, methylene blue, new methylene blue, or Leishman smear staining). Typically, the brightfield image that was acquired under violet lighting conditions is used in the aforementioned manner, since violet light is absorbed strongly by hemoglobin and therefore red blood cells appear as red once the contrast of the image is made negative and the image is mapped to the red channel.

For some applications, data from each of the plurality of brightfield images acquired using respective brightfield imaging modalities are combined such as to generate an artificial color microscopic image of the microscopic imaging field that appears like a color smear image. Typically, at least one of the brightfield images is acquired under violet lighting conditions, as described hereinabove.

For some applications, three images are acquired under respective imaging modalities. For example, the image acquired under brightfield violet lighting conditions is combined (a) with two additional brightfield images, (b) with an additional brightfield image and a fluorescent image, or (c) with two fluorescent images. For example, two fluorescent images may be acquired after exciting the blood sample with light at respective wavelength bands. Alternatively, the two fluorescent images may be acquired after exciting the sample with light at the same wavelength band, but using respective, different emission filters.

Typically, the second image is mapped to a second color channel of the artificial color microscopic image, and the third image is mapped to a third color channel of the artificial color microscopic image. For example, when an RGB color model is used, the first image may be mapped to the red channel (as described above), the second image mapped to the green channel, and the third image mapped to the blue channel. For some applications, one of the second and third images is acquired while the sample is excited using light (e.g., UV light) that causes cell nuclei (e.g., DNA of the cell nuclei) to fluoresce. Alternatively or additionally, a second one of the second and third images is acquired while the sample is excited using light (e.g., blue light) that causes RNA and/or cytoplasm to fluoresce. For some applications, imaging modalities are used that are similar to those used in images generated using Giemsa, Romanowsky-Giemsa, Wright-Romanowsky, Jenner, Wright, Field, May-Grünwald, methylene blue, new methylene blue, or Leishman smear staining. Typically, the image that is generated has a similar appearance to that of a color smear image (e.g., similar to an image generated using Giemsa, Romanowsky-Giemsa, Wright-Romanowsky, Jenner, Wright, Field, May-Grünwald, methylene blue, new methylene blue, or Leishman smear staining).

For some applications, each of the fluorescent images is acquired using a relatively long exposure time. For example, this may be used in order to visualize reticulocytes as well as platelets. Alternatively, one of the fluorescent images may be acquired using a relatively short exposure time, and the other one of the fluorescent images may be acquired using a relatively long exposure time. The long and short exposure fluorescent images typically contain different information. The images acquired using the short exposure are typically optimized to provide data relating to white blood cells and other high intensity objects, while the images acquired using the long exposure time are typically optimized to provide data relating to low intensity objects such as reticulocytes, platelets, parasites, ghost cells, etc.

For some applications, the short-exposure-time images are combined with the long-exposure-time images into a single fluorescent image (for example, by replacing over-exposed regions in the long-exposure-time image with the corresponding region in the short-exposure-time image). For some applications, the resultant composite image (and/or a composite image that is generated using a different composite-image-generation technique) is mapped to one of the channels of an artificial color images, e.g., using the techniques described hereinabove.

For some applications, a plurality of images are mapped to a single channel. For example, two or more brightfield images, which are acquired under respective imaging modalities may be mapped to one of the channels. Alternatively, two or more fluorescent images, which are acquired under respective imaging modalities may be mapped to one of the channels. Further alternatively, a combination of one or more brightfield images and one or more fluorescent images may be mapped to a single channel. For some applications, a plurality of images are mapped to each of a plurality of the channels in the above-described manner.

For some applications, a neural network is used in the generation of an artificial color image. In some cases, an artificial color image generated using the methods described hereinabove may have different characteristics from the type of images that are commonly used in the field. For example, such images may differ from standard images in color, intensity resolution, shading, etc. For some applications, a convolutional neural network is used to generate an image that is more similar to standard images in the field, such that the image has a similar appearance to that of a color smear image (e.g., similar to an image generated using Giemsa, Romanowsky-Giemsa, Wright-Romanowsky, Jenner, Wright, Field, May-Grünwald, methylene blue, new methylene blue, or Leishman smear staining).

For some applications, one or more of the images that are mapped to the color image is normalized. For example, the image may be normalized by dividing the image by a background map. Alternatively or additionally, a function of the image (such as optical density) may be used in the color image. For some applications, the displayed color image is normalized such that the relevant features are of similar magnitude in all of the channels. For some applications, one or more of the original images, and/or the displayed color image is normalized by determining a maximum intensity within the image, and removing all pixels having an intensity that is less than a given proportion of the maximum intensity (e.g., less than half of the maximum intensity), and renormalizing the pixel intensity as described below. For some applications, one or more of the original images, and/or the displayed color image is normalized in the following manner. An intensity histogram of the image is generated. For each pixel within the image that has an intensity that is at least equal to half of the maximum intensity, a closest local maximum in the intensity histogram having an intensity that is greater than half of the maximum intensity within the image is identified. The intensity of the pixel is then normalized based upon the difference between the maximum intensity and the intensity of the local maximum. For example, a given pixel may be assigned an intensity based upon the following formula:

$$INp = N * ( Ip - V min ) / ( V min - V max ) , for V min <= Ip <= V max ;$$

$$INp = N for Ip > V max ;$$

$$INp = 0 for Ip < V min$$

where:
INp is the normalized intensity of the pixel,
N is an integer (e.g., 255),
Ip is the original intensity of the pixel,
Vmax is the maximum intensity within the image, and
V min is the intensity of the closest maximum having an intensity that is greater than half of the maximum intensity within the image.

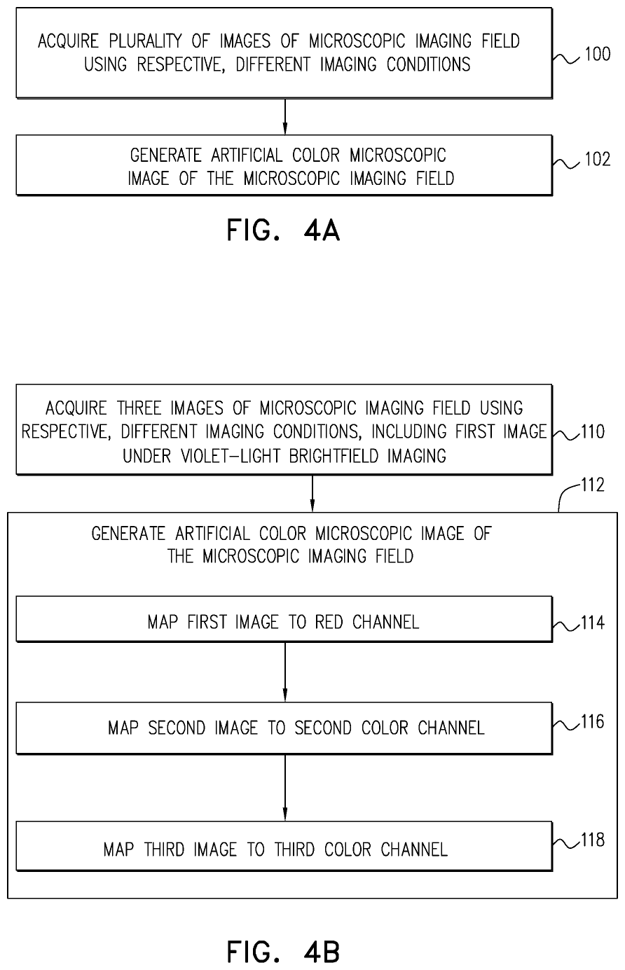

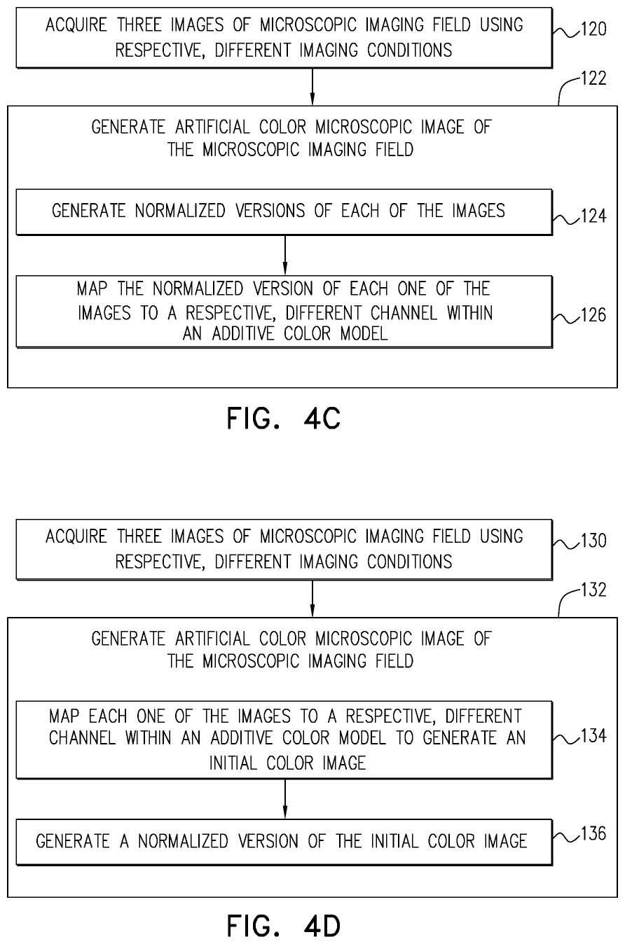

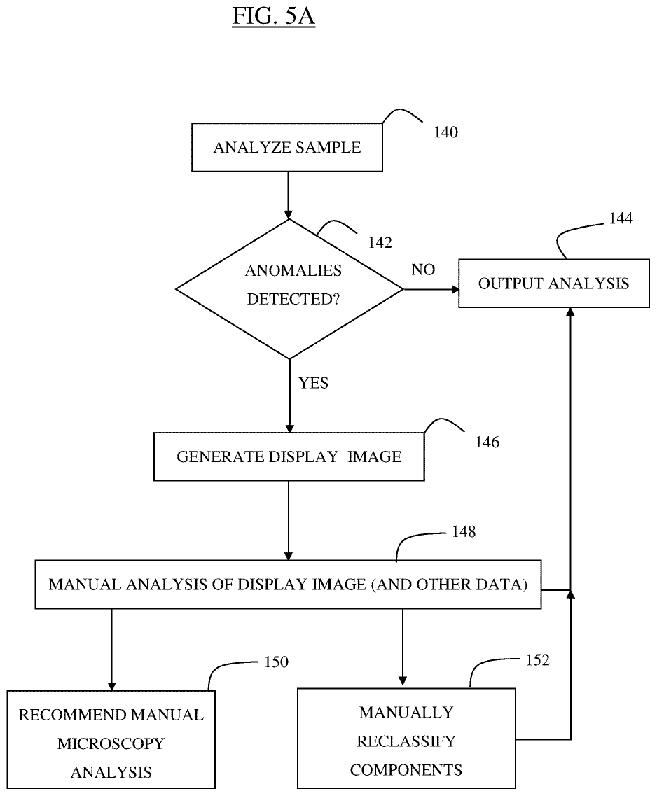

Reference is now made to FIGS. 4A-D, which are flowcharts showing steps of methods that are performed, in accordance with some applications of the present invention, in accordance with the techniques described hereinabove.

Referring to FIG. 4A, for some applications, in step 100, a plurality of images of a microscopic imaging field of the blood sample are acquired, each of the images being acquired using respective, different imaging conditions. Subsequently, in step 102, data from each of the plurality of images are combined such as to generate an artificial color microscopic image of the microscopic imaging field that appears like a color smear image. Step 102 is typically performed by computer processor 28.

Referring to FIG. 4B, for some applications, in step 110, three images of a microscopic imaging field of the blood sample are acquired using the microscope, each of the images being acquired using respective, different imaging conditions, and the first one of the three images being acquired under violet-light brightfield imaging. Subsequently, in step 112, an artificial color microscopic image of the microscopic imaging field is generated, by mapping the first one of the three images to a red channel of the artificial color microscopic image (sub-step 114), mapping a second one of the three images to a second color channel of the artificial color microscopic image (sub-step 116), and mapping a third one of the three images to a third color channel of the artificial color microscopic image (sub-step 118). Step 112, and sub-steps 114-118, are typically performed by computer processor 28.

Referring to FIG. 4C, for some applications, in step 120, three images of a microscopic imaging field of the blood sample are acquired using the microscope, each of the images being acquired using respective, different imaging conditions. Subsequently, in step 122, an artificial color microscopic image of the microscopic imaging field is generated, by generating normalized versions of each of the images, such as to remove pixels within the image having an intensity that is below a threshold (sub-step 124), and mapping the normalized version of each one of the images to a respective, different channel within an additive color model (sub-step 126). Step 122, and sub-steps 124-126, are typically performed by computer processor 28.

Referring to FIG. 4D, for some applications, in step 130, three images of a microscopic imaging field of the blood sample are acquired using the microscope, each of the images being acquired using respective, different imaging conditions. Subsequently, in step 132, an artificial color microscopic image of the microscopic imaging field is generated, by mapping each one of the images to a respective, different channel within an additive color model to generate an initial color image (sub-step 134), and generating a normalized version of the initial color image, such as to remove pixels within the image having an intensity that is below a threshold (sub-step 136). Step 132, and sub-steps 134-136, are typically performed by computer processor 28.

Reference is now made to FIG. 5A, which is a flowchart showing steps of a workflow that is performed in accordance with some applications of the present invention. Reference is also made to FIG. 5B, which is a block diagram indicating steps that are performed by respective portions of a bodily-sample analysis system, in accordance with some applications of the present invention. As described hereinabove, typically, computer processor 28 analyzes the microscopic images and/or other data relating to the sample (e.g., optical absorption measurements, scattering measurements, and/or spectroscopic measurements), in order to determine properties of the sample. Typically, the computer processor analyzes a plurality of different microscopic images that are acquired using respective imaging modalities. For example, the microscope images may be acquired using fluorescent and brightfield imaging modalities as described hereinabove. For some applications, the computer processor is configured to automatically identify components in the blood such as platelets, white blood cells, anomalous white blood cells, circulating tumor cells, red blood cells, reticulocytes, Howell-Jolly bodies, etc. For some applications, the computer processor is configured to determine parameters relating to one or more of the components. For example, in relation to red blood cells, the computer processor determines parameters such as corpuscular hemoglobin (MCH), mean corpuscular volume (MCV), red blood cell distribution width (RDW), red blood cell morphologic features, clumping, and/or red blood cell abnormalities. Alternatively or additionally, in relation to white blood cells, the computer processor determines parameters such as absolute and relative numbers of neutrophils, lymphocytes, monocytes, eosinophils and basophils. For some applications, the computer processor performs normal and abnormal leukocyte differentiation, including detecting the existence of immature or hyper segmented cells, white blood cell agglutination or fragmentation, blasts, and/or atypical or abnormal lymphocytes. For some applications, the computer processor detects leukocyte subpopulations (such as B,T-cells), and/or morphological cell activation, and/or or any other cell or population based biomarkers (e.g. monocyte distribution width (MDW). Further alternatively or additionally, in relation to platelets the computer processor determines parameters such as the presence of giant platelets, platelets clumps or abnormal platelets distribution, immature (i.e., reticulated) platelets fraction, average platelet size (MPV), platelet distribution width (PDW), platelet clumping, and/or platelet activation levels. For some applications, the computer processor detects parasites, bacteria, fungi, and/or any other abnormal biomarkers. For some applications, the computer processor determines parameters such as the relative number of blast cells, nucleated red blood cells, abnormal or atypical lymphocyte cells, immature granulocyte cells and/or reticulocytes.

Referring to FIG. 5A, for some applications, in a first step 140, computer processor 28 (which is disposed within or is directly connected to the optical measurement unit) performs the above-described analysis of a sample. If the computer processor performs the analysis and does not detect any anomalies within the sample (in step 142), then the computer processor generates an output based on the analysis (step 144), which in the case of a blood sample will typically be a complete blood count report. In such cases, the computer processor typically does not generate a display image of the sample (e.g. using the techniques described with reference to FIGS. 4A-D). For some applications, in response to detecting an anomaly within the sample (in step 142), then, in step 146, a display image of the sample is generated (e.g. using the techniques described with reference to FIGS. 4A-D). As described in further detail hereinbelow, a display image is typically an image that combines microscopic images acquired using respective imaging modalities and that is displayed to a user for analysis, rather than a raw microscopic image of the type that is analyzed by the computer processor. Typically, computer processor 28 sends data to remote computer server 29 and the remote computer processor generates the display image using the data, as indicated in FIG. 5B. Alternatively, the display image is generated by computer processor 28, and is sent to the remote computer network.

For some applications, step 146 is performed in response to detecting a distributional anomaly (i.e., an anomaly relating to the number of a type of entity distributed within the sample). Alternatively or additionally, step 146 is performed in response to detecting a morphological anomaly (i.e., an anomaly relating to one or more occurrences of a given morphological entity or phenomenon within the sample). For some applications, in response to detecting an anomaly, the sample is first flagged, such that the user can select whether they wish to see an image of the sample. For some applications, an image is generated in which the anomaly that gave rise to the image being generated is displayed. For some applications, if the anomaly that was detected was a concentration of a given entity that is high (e.g., greater than a threshold concentration), then an image of that entity may be generated, or an image in which that entity is highlighted may be generated. For example, if the anomaly that was detected was a large number of platelet clumps (e.g., a concentration of platelet clumps that is greater than a threshold concentration), then an image of the platelets clumps may be generated, or an image in which platelet clumps are highlighted may be generated. Alternatively or additionally, if the anomaly that was detected was a large number of blast cells (e.g., a concentration of blast cells) that is greater than a threshold concentration), then an image of the blast cells may be generated, or an image in which blast cells are highlighted may be generated.

For some applications, step 146 is performed in response to detecting one or more of the following entities (and/or more than a given concentration thereof): platelet clumps, giant platelets, enlarged platelets, blast cell, immature granulocytes, atypical lymphocytes, abnormal lymphocytes, nucleated red blood cells, schistocytes, sickle cells, target cells, tear drop cells, Howell Jolly bodies, Pappenheimer bodies, basophilic stippling, and/or parasites. For some applications, step 146 is performed in response to detecting a color abnormality, (e.g., a red blood cell color abnormality, for example, polychromatic cells and/or hypochromatic cells), a size abnormality (e.g., macrocytosis, microcytosis, and/or anisocytosis), and/or a shape abnormality (e.g., poikilocytosis, target cells, schistocytes, helmet cells, sickle cells, spherocytes, elliptocytes, ovalocytes, tear drop cells, stomatocytes, acanthocytes, and/or echinocytes).

It is noted that typically, in step 146, the display image is generated using the same data that are used to perform the analysis of the sample in step 140. For example, the techniques described with reference to FIGS. 4A-D may be used to generate the display image. Typically, the display image is formed by combining a plurality of microscopic images that were acquired using respective imaging modalities, such as the brightfield and/or fluorescent imaging modalities described hereinabove. Typically, the image that is generated has a similar appearance to that of a color smear image (e.g., similar to an image generated using Giemsa, Romanowsky-Giemsa, Wright-Romanowsky, Jenner, Wright, Field, May—Grünwald, methylene blue, new methylene blue, or Leishman smear staining), but without requiring the sample to be smeared and/or stained. This is in contrast to alternative techniques, whereby in response to an optical measurement unit detecting an anomaly within the sample, the subject is instructed to take a further sample of blood and image the further sample with a different optical measurement system (that typically requires the sample to be smeared and/or stained) in order to generate a display image of the blood. Rather, in accordance with the present disclosure, a display image is generated using the same data and the same sample as were analyzed in step 140. However in step 146, those data are used to generate a display image rather than being analyzed to generate a report. Typically, this is less cumbersome for the subject and saves resources relative to the alternative techniques. For example, it is not necessary for a clinic to include two separate analysis systems, i.e., a first analysis system for performing the initial analysis and a second analysis system for imaging the sample, in response to the first system detecting an anomaly within the sample. In addition, the subject is only required to take one sample, and that same sample is used both for performing the initial analysis and for generating the display image. It is noted however, that typically the display image is only generated in response to computer processor 28 detecting an anomaly within the sample. This is because, although all of the data that are required for generating the display image have typically already been acquired in step 140, it requires additional computational resources to generate the display image. Therefore, the step of generating the display image (i.e., step 146) is only performed if a need is identified, such as in the event of computer processor 28 detecting an anomaly within the sample. For some applications, in order to generate the display image, some cells are reimaged, for example, in order to create an image of certain cells, at a better exposure for analysis by the user.

As described hereinabove, for some applications, the step of generating the display image is performed at computer processor 28. Alternatively, the data for generating the display image are communicated to remote computer server 29, and the display image generated at the remote computer server. Typically, computer processor 28 communicates to the remote computer server the outcome of the analysis that was performed in step 140. In particular, computer processor typically communicates an indication of the anomaly within the sample that was identified. Thus, at the remote computer server, the display image as well as the indication of the anomaly within the sample that was identified are typically stored. For some applications, the patient's medical records and/or past blood (or other bodily sample) analyses are also stored at, and/or are accessible via, the remote computer server.

Typically, in step 148, a user (such as a trained laboratory technician) analyzes the data relating to the subject, including the display image, the outcome of the analysis that was performed in step 140, and, optionally, the subject's medical records and/or past blood (or other bodily sample) analyses. For some applications, the user receive additional data, such as information relating to the instrument performance (e.g., quality control results and/or failsafe or error flags that have been generated by the instrument). For some applications, the user analyzes at least some of the aforementioned data via the optical measurement device, e.g., via user interface 32. Typically, the user analyzes at least some of the aforementioned data via the remote computer network. For example, the optical measurement unit (with computer processor 28) may be located at a clinic or at a pharmacy, and the remote computer server may be at a center in which trained laboratory technicians are located. As described with reference to FIG. 1, for some applications, remote user interface 33 and/or remote output device 35 is itself remote from the remote computer server. Thus, for example, the optical measurement unit (with computer processor 28) may be located at a clinic or at a pharmacy, and the remote user interface 33 and/or remote output device 35 may be a remote user's (e.g., the remote trained laboratory technician's) computer, tablet device, smartphone, etc. In such cases, the remote computer server may generate an alert at the remote user interface or the remote output device indicating that her/his analysis of a sample is required. For some applications, more than one user may analyze the data in step 148. For example, a junior and a senior trained laboratory technician may both access the data using a remote user interface.

Typically, in response to the user (e.g., the remote trained laboratory technician) determining that the anomaly that was identified within the sample is not a concern, the results of the analysis that was performed in step 140 (e.g., a complete blood count) is outputted to the subject (e.g., via user interface 32, or output device 34), and/or released such that it is available within a medical information system. Further typically, if there is a concern regarding the sample, then the user (e.g., the remote trained laboratory technician) can indicate that the subject should undergo a regular manual microscopy examination (step 150). Typically, in such cases the user (e.g., the remote trained laboratory technician) will communicate this to the subject via the remote computer server, with the output itself typically being generated at the optical measurement unit 31. For some applications, in step 152, the user (e.g., the remote trained laboratory technician) can reclassify components within the sample. In particular, in some cases, certain components that were flagged in step 140 may be reclassified by the user.

Referring to FIG. 5B, typically the steps of automatically analyzing the sample, identifying and analyzing components in the sample, and identifying anomalies in the sample are performed by computer processor 28, which is disposed within and/or in disposed locally with respect to the optical measurement unit. For example, the optical measurement unit (with computer processor 28) may be located at a clinic or at a pharmacy. For some applications, if it is determined that a display image should be generated, then this step is performed at remote computer server 29. Alternatively, as described hereinabove, the display image may be generated at computer processor 28. As described hereinabove, for some applications, the remote computer processor also provides access to additional data such as the analysis of sample, anomalies that were detected within the sample, the subject's medical history, information relating to the instrument performance (e.g., quality control results and/or failsafe or error flags that have been generated by the instrument), and/or previous sample analysis of the subject (e.g., by storing the additional data in a memory). For some applications, the remote computer server generates an alert. For example, in response to detecting that a sample requires manual analysis, the remote computer server may generate an alert at remote user interface 33 and/or remote output device 35. As described hereinabove, the remote user interface 33 and/or remote output device 35 may be a remote user's (e.g., the remote trained laboratory technician's) computer, tablet device, smartphone, etc. Typically, the remote user interface is used to allow the remote user to perform one or more of the following actions: accessing the display image and additional data, reclassifying components within the sample, validating the automated sample analysis, and/or recommending manual microscopy analysis. For some applications, an output is generated by computer processor 28 based on any one of the aforementioned steps. For example, the computer processor may output the results of the automated sample analysis, computer processor 28 may generate an output indicating that the sample has been sent for further processing, and/or computer processor 28 may generate an output indicating that it is recommended that the subject has their bodily sample analyzed using manual microscopy analysis.

### EXAMPLE

An example of the use of microscopic images that are acquired using the techniques described herein in order to generate an image that can be interpreted by a microscopist in order to identify anomalous features is now described.

In this study, a batch of blood samples (containing normal and abnormal samples) were analyzed using an optical measurement unit as described herein. A portion of the samples had high counts of certain white blood cells relative to the total number, which were defined as distributional anomalies according to the following thresholds:
Neutrophils more than 80 percent;
Lymphocytes more than 50 percent;
Monocytes more than 14 percent;
Eosinophils more than 7 percent;
Basophils more than 2.5 percent.

Images of the samples were generated using the techniques described hereinabove, and the images were analyzed by two qualified microscopists. In addition, for each sample, two blood films were prepared and stained within 2 hours of being scanned by the optical measurement unit. Each of the blood films was evaluated by the same two qualified microscopists using a manual microscope. The microscopists each performed a 200-cell differential count (i.e., 400 cells counted per sample in total). In addition to other parameters, the microscopists recorded findings of percentages of neutrophils, lymphocytes, monocytes, eosinophils, and basophils relative to the total number of white blood cells within the sample using images generated using the techniques described herein and using manual microscopic evaluation of the blood films. Regression analysis was performed on the results as detected by the microscopists using images generated using the techniques described herein versus the results as detected by the microscopists using manual microscopic evaluation of the blood films, with the results being shown in Table 1 below:

TABLE 1
Relationship between entities detected by the microscopists
using images generated using the techniques described herein
versus the results as detected by the microscopists using
manual microscopic evaluation of the blood films.

| | Number of | Percentage | Correlation | |
|---|---|---|---|---|
| Entity | samples | range | coefficient | Slope |
| Neutrophils | 37 | 49.8 to 92.3 | 0.953 | 0.88 |
| Lymphocytes | 36 | 2.8 to 38.8 | 0.950 | 0.90 |
| Monocytes | 37 | 1.8 to 21.2 | 0.863 | 0.98 |
| Eosinophils | 37 | 0.0 to 4.8 | 0.891 | 1.12 |
| Basophils | 37 | 0.0 to 1.8 | 0.541 | 1.00 |

As can be seen from Table 1, for each of the neutrophils, lymphocytes, monocytes, and eosinophils the correlation between the findings of the microscopists using images generated using the techniques described herein versus the findings as detected by the microscopists using manual microscopic evaluation of the blood films is greater than 0.85. For the basophils the correlation was only 0.541. Nevertheless, these results indicate that images generated using the techniques described herein may be suitable for detection of entities within a blood sample.

It also follows from the data shown in Table 1 that distributional anomalies may be detected using images generated using the techniques described herein. For example, among neutrophils and monocytes, there was good correlation between the results even when the percentage ranges for these entities reached a level that was predefined as indicating a distributional anomaly. Similarly, morphological anomalies may be detected using images generated using the techniques described herein, by training microscopists to identify such morphological anomalies within images generated using the techniques described herein.

For some applications, the apparatus and methods described herein are applied to a bodily sample, such as, blood, urine, peritoneal fluid, cerebrospinal fluid, saliva, semen, sweat, sputum, vaginal fluid, stool, breast milk, bronchoalveolar lavage, gastric lavage, tears and/or nasal discharge, mutatis mutandis. The bodily sample may be from any living creature, and is typically from warm blooded animals. For some applications, the bodily sample is a sample from a mammal, e.g., from a human body. For some applications, the sample is taken from any domestic animal, zoo animals and farm animals, including but not limited to dogs, cats, horses, cows and sheep. Alternatively or additionally, the bodily sample is taken from animals that act as disease vectors including deer or rats.

For some applications, the apparatus and methods described herein are applied to a non-bodily sample. For some applications, the sample is an environmental sample, such as, a water (e.g. groundwater) sample, surface swab, soil sample, air sample, or any combination thereof, mutatis mutandis. In some embodiments, the sample is a food sample, such as, a meat sample, dairy sample, water sample, wash-liquid sample, beverage sample, and/or any combination thereof.

For some applications, the sample as described herein is a sample that includes blood or components thereof (e.g., a diluted or non-diluted whole blood sample, a sample including predominantly red blood cells, or a diluted sample including predominantly red blood cells), and parameters are determined relating to components in the blood such as platelets, white blood cells, anomalous white blood cells, circulating tumor cells, red blood cells, reticulocytes, Howell-Jolly bodies, etc.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purposes of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 28) coupled directly or indirectly to memory elements (e.g., memory 30) through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described herein, computer processor 28 typically acts as a special purpose artificial-image-generation computer processor. Typically, the operations described herein that are performed by computer processor 28 transform the physical state of memory 30, which is a real physical article, to have a different magnetic polarity, electrical charge, or the like depending on the technology of the memory that is used.

The apparatus and methods described herein may be used in conjunction with apparatus and methods described in any one of the following patents or patent applications, all of which are incorporated herein by reference:
U.S. Pat. No. 9,522,396 to Bachelet;
U.S. Pat. No. 10,176,565 to Greenfield;
U.S. Pat. No. 10,640,807 to Pollak;
U.S. Pat. No. 9,329,129 to Pollak;
U.S. Pat. No. 10,093,957 to Pollak;
U.S. Pat. No. 10,831,013 to Yorav Raphael;
U.S. Pat. No. 10,843,190 to Bachelet;
U.S. Pat. No. 10,482,595 to Yorav Raphael;
U.S. Pat. No. 10,488,644 to Eshel;
U.S. Pat. No. 11,733,150 to Eshel;
U.S. Pat. No. 11,307,196 to Pollak;
U.S. Pat. No. 11,099,175 to Zait;
U.S. Pat. No. 11,614,609 to Yorav-Raphael;
WO 21/079305 to Pecker;
WO 21/079306 to Pecker;
WO 21/116955 to Yafin;
WO 21/116957 to Gluck;
WO 21/116959 to Eshel;
WO 21/116960 to Zait;
WO 21/116962 to Halperin;
WO 22/009104 to Zait; and
WO 23/144713 to Pecker.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for use with a bodily sample, the method comprising:
using a microscope, acquiring a plurality microscopic images of the sample, using respective imaging modalities; and
using at least one computer processor:
analyzing the microscopic images to identify and classify components within the bodily sample;
detecting whether there are any anomalies within the sample that require further analysis;
in response to detecting that there are anomalies within the sample that require further analysis:
generating a display image that combines the images that were acquired using respective imaging modalities; and
driving a display to display the display image to a user; and
in response to detecting that there are not anomalies within the sample that require further analysis, generating an output based upon the analysis of the microscopic images, without generating a display image that combines images that were acquired using respective imaging modalities.

2-4. (canceled)

5. The method according to claim 1, wherein generating the display image that combines the images that were acquired using respective imaging modalities comprises generating an image having a similar appearance to that of a color smear image without requiring the sample to be smeared and/or stained.

6-7. (canceled)

8. The method according to claim 1, wherein detecting the anomaly comprises detecting an indication that a concentration of a given entity within the sample is greater than a threshold amount, and wherein generating the display image comprises generating a display image showing the given entity and/or generating a display image in which the given entity is highlighted.

9. (canceled)

10. The method according to claim 1, wherein the bodily sample includes a blood sample and detecting the anomaly comprises detecting an indication of a presence, or a concentration that exceeds a threshold, of giant platelets, platelet clumps, and/or enlarged platelets.

11. (canceled)

12. The method according to claim 1, wherein the bodily sample includes a blood sample and detecting the anomaly comprises detecting an indication of a presence, or a concentration that exceeds a threshold, of blast cells, immature granulocytes, atypical lymphocytes, and/or abnormal lymphocytes.

13. (canceled)

14. The method according to claim 1, wherein the bodily sample includes a blood sample and detecting the anomaly comprises detecting an indication of a presence, or a concentration that exceeds a threshold, of nucleated red blood cells, schistocytes, sickle cells, target cells, and/or tear drop cells.

15. (canceled)

16. The method according to claim 1, wherein the bodily sample includes a blood sample and detecting the anomaly comprises detecting an indication of a presence, or a concentration that exceeds a threshold, of polychromatic red blood cells and/or hypochromatic red blood cells.

17. (canceled)

18. The method according to claim 1, wherein the bodily sample includes a blood sample and detecting the anomaly comprises detecting an indication of a presence, or a concentration that exceeds a threshold, of red blood cell macrocytosis, microcytosis, and/or anisocytosis.

19. (canceled)

20. The method according to claim 1, wherein the bodily sample includes a blood sample and detecting the anomaly comprises detecting an indication of a presence, or a concentration that exceeds a threshold, of poikilocytosis, target cells, schistocytes, helmet cells, sickle cells, spherocytes, elliptocytes, ovalocytes, tear drop cells, stomatocytes, acanthocytes, and/or echinocytes.

21. Apparatus for use with a bodily sample, the apparatus comprising:
a microscope configured to acquire a plurality microscopic images of the sample, using respective imaging modalities; and
at least one computer processor configured to:
analyze the microscopic images to identify and classify components within the bodily sample;
detect whether there are any anomalies within the sample that require further analysis;
in response to detecting that there are anomalies within the sample that require further analysis:
generate a display image that combines the images that were acquired using respective imaging modalities; and
drive a display to display the display image to a user; and
in response to detecting that there are not anomalies within the sample that require further analysis, generate an output based upon the analysis of the microscopic images, without generating a display image that combines images that were acquired using respective imaging modalities.

22-23. (canceled)

24. The apparatus according to claim 21, wherein the at least one computer processor is configured:
in response to detecting that there are anomalies within the sample that require further analysis, to output an indication of the anomalies to the user, and
to generate the display image that combines the images that were acquired using respective imaging modalities in response to receiving an input from the user indicating that the display image should be generated.

25. The apparatus according to claim 21, wherein the at least one computer processor is configured to generate the display image that combines the images that were acquired using respective imaging modalities by generating an image having a similar appearance to that of a color smear image without requiring the sample to be smeared and/or stained.

26-27. (canceled)

28. The apparatus according to claim 21, wherein the at least one computer processor is configured to detect an indication that a concentration of a given entity within the sample is greater than a threshold amount, and wherein the at least one computer processor is configured to generate display image showing the given entity and/or generating a display image in which the given entity is highlighted.

29. (canceled)

30. The apparatus according to claim 21, wherein the bodily sample includes a blood sample and the at least one computer processor is configured to detect an indication of a presence, or a concentration that exceeds a threshold, of giant platelets, platelet clumps, and/or enlarged platelets.

31. (canceled)

32. The apparatus according to claim 21, wherein the bodily sample includes a blood sample and the at least one computer processor is configured to detect an indication of a presence, or a concentration that exceeds a threshold, of blast cells, immature granulocytes, atypical lymphocytes, and/or abnormal lymphocytes.

33. (canceled)

34. The apparatus according to claim 21, wherein the bodily sample includes a blood sample and the at least one computer processor is configured to detect an indication of a presence, or a concentration that exceeds a threshold, of nucleated red blood cells, schistocytes, sickle cells, target cells, and/or tear drop cells.

35. (canceled)

36. The apparatus according to claim 21, wherein the bodily sample includes a blood sample and the at least one computer processor is configured to detect an indication of a presence, or a concentration that exceeds a threshold, of polychromatic red blood cells and/or hypochromatic red blood cells.

37. (canceled)

38. The apparatus according to claim 21, wherein the bodily sample includes a blood sample and the at least one computer processor is configured to detect an indication of a presence, or a concentration that exceeds a threshold, of red blood cell macrocytosis, microcytosis, and/or anisocytosis.

39. (canceled)

40. The apparatus according to claim 21, wherein the bodily sample includes a blood sample and the at least one computer processor is configured to detect an indication of a presence, or a concentration that exceeds a threshold, of poikilocytosis, target cells, schistocytes, helmet cells, sickle cells, spherocytes, elliptocytes, ovalocytes, tear drop cells, stomatocytes, acanthocytes, and/or echinocytes.

41. A computer software product, for use with a plurality microscopic images of a bodily sample, each of the microscopic images having been acquired using respective imaging modalities, the computer software product comprising a non-transitory computer-readable medium in which program instructions are stored, which instructions, when read by a computer cause the computer to perform the steps of:
analyzing the microscopic images to identify and classify components within the bodily sample;
detecting whether there are any anomalies within the sample that require further analysis;
in response to detecting that there are anomalies within the sample that require further analysis:
generating a display image that combines the images that were acquired using respective imaging modalities; and
driving a display to display the display image to a user; and
in response to detecting that there are not anomalies within the sample that require further analysis, generating an output based upon the analysis of the microscopic images, without generating a display image that combines images that were acquired using respective imaging modalities.
